(19) United States
(12) Patent Application Publication
(10) Pub. No.: US 2008/0234305 A1
(43) Pub. Date: Sep. 25, 2008

(54) Novel Tetrahydropyridine Derivatives

(76) Inventors: Olivier Bezencon, Riehen (CH); Daniel Bur, Therwil (CH); Walter Fischli, Allschwil (CH); Lubos Remen, Allschwil (CH); Sylvia Richard-Bildstein, Dietwiller (FR); Thierry Sifferlen, Guewenheim (FR); Thomas Weller, Binningen (CH)

Correspondence Address: Thomas Hoxie;Hoxie & Tso, 374 Millburn Avenue, Suite 300E, Millburn, NJ, 07041, US

(21) Appl. No.: 10/575,348

(22) PCT Filed: Oct. 5, 2004
(86) PCT No.: PCT/EP04/11088

§ 371 Date: Apr. 7, 2006

(30) Foreign Application Priority Data
Oct. 9, 2003 (EP) PCT/EP03/11146

(51) Int. Cl. A61K 31/438; C07D 221/20; C07D 213/80; A61K 31/44; A61P 9/04

(52) U.S. Cl. 514/278; 546/16; 514/356; 546/322

(57) ABSTRACT

The invention relates to novel tetrahydropyridine derivatives and use thereof as active ingredients in the preparation of pharmaceutical compositions. The invention also concerns related aspects including processes for the preparation of the compounds, pharmaceutical compositions containing one or more of those compounds and especially their use as inhibitors of renin. (I) wherein X and Y represent independently hydrogen, fluorine or a methyl group; X and Y do not represent both hydrogen at the same time or X and Y may together form a cyclopropyl ring; W represents a phenyl or a heteroaryl, the heteroaryl ring being a six-membered and non-fused ring, the phenyl ring and the heteroaryl are substituted with V in position 3 or 4; A and B independently represent —O—; —S—; —SO— or —SO₂—; U represents aryl or heteroaryl; T represents —CONR¹—; —(CH₂)ₚOCO—; —(CH₂)ₚN(R¹)CO—; —(CH₂)ₚN(R¹)SO₂—; —COO—; —(CH₂)ₚOCONR¹— or —(CH₂)ₚN(R²)CONR¹—; R¹ and R² independently represent hydrogen; lower alkyl; lower alkenyl; lower alkynil; cycloalkyl; aryl-lower alkyl, heteroaryl-lower alkyl or cycloalkyl-lower alkyl; Q represents lower alkylene or lower alkenylene; M represents hydrogen; cycloalkyl; aryl; heterocyclyl or heteroaryl:

The invention relates to novel five-membered heteroaryl derivatives of the general formula (I). The invention also concerns related aspects including processes for the preparation of the compounds, pharmaceutical compositions containing one or more compounds of formula (I) and especially their use as renin inhibitors in cardiovascular events and renal insufficiency.

In the renin-angiotensin system (RAS) the biologically active angiotensin II (Ang II) is generated by a two-step mechanism. The highly specific enzyme renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAS represents a major advance in the treatment of cardiovascular diseases. ACE inhibitors and AT₁ blockers have been accepted to treat hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al., Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N. Engl. J. Med., 1992, 327, 669).

The rationale to develop renin inhibitors is the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be by-passed by chymase, a serine protease (Husain A., J. Hypertens., 1993, 11, 1155). In patients inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). ACE inhibitors do not inhibit Chymase. Therefore, the formation of Ang II is still possible in patients treated with ACE inhibitors. Blockade of the AT₁ receptor (e.g. by losartan) on the other hand overexposes other AT-receptor subtypes (e.g. AT₂) to Ang II, whose concentration is significantly increased by the blockade of AT₁ receptors. In summary, renin inhibitors are expected to demonstrate a different pharmaceutical profile than ACE inhibitors and AT₁ blockers with regard to efficacy in blocking the RAS and in safety aspects.

Only limited clinical experience (Azizi M. et al., J. Hypertens., 1994, 12, 419; Neutel J. M. et al., Am. Heart, 1991, 122, 1094) has been created with renin inhibitors because of their insufficient oral activity due to their peptidomimetic character (Kleinert H. D., Cardiovasc. Drugs, 1995, 9, 645). The clinical development of several compounds has been stopped because of this problem together with the high cost of goods. Only one compound containing four chiral centers has entered clinical trials (Rahuel J. et al., Chem. Biol., 2000, 7, 493; Mealy N. E., Drugs of the Future, 2001, 26, 1139). Thus, renin inhibitors with good oral bioavailability and long duration of action are required. Recently, the first non-peptide renin inhibitors were described which show high in vitro activity (Oefner C. et al., Chem. Biol., 1999, 6, 127; Patent Application WO97/09311; Märki H. P. et al., II Farmaco, 2001, 56, 21). However, the development status of these compounds is not known.

The present invention relates to the identification of renin inhibitors of a non-peptidic nature and of low molecular weight. Described are orally active renin inhibitors of long duration of action, which are active in indications beyond blood pressure regulation where the tissular renin-chymase system may be activated leading to pathophysiologically alter local functions such as renal, cardiac and vascular remodeling, atherosclerosis, and possibly restenosis. So, the present invention describes these non-peptidic renin inhibitors.

The following paragraphs provide definitions of the various chemical moieties that make up the compounds according to the invention and are intended to apply uniformly throughout the specification and claims unless an otherwise expressly set out definition provides a broader definition.

The term lower alkyl, alone or in combination with other groups, means saturated, straight and branched chain groups with one to seven carbon atoms, preferably one to four carbon atoms that can be optionally substituted by halogens. Examples of lower alkyl groups are methyl, ethyl, n-propyl, iso-propyl, n-butyl, iso-butyl, sec-butyl, tert-butyl, pentyl, hexyl and heptyl. The methyl, ethyl and isopropyl groups are preferred.

The term lower alkoxy refers to a R—O group, wherein R is a lower alkyl. Examples of lower alkoxy groups are methoxy, ethoxy, propoxy, iso-propoxy, iso-butoxy, sec-butoxy and tert-butoxy.

The term lower alkenyl, alone or in combination with other groups, means straight and branched chain groups comprising an olefinic bond and two to seven carbon atoms, preferably two to four carbon atoms, that can be optionally substituted by halogens. Examples of lower alkenyl are vinyl, propenyl or butenyl.

The term lower alkinyl, alone or in combination with other groups, means straight and branched chain groups comprising a triple bond and two to seven carbon atoms, preferably two to four carbon atoms that can be optionally substituted by halogens. Examples of lower alkinyl are ethinyl, propinyl or butinyl.

The term lower alkylene, alone or in combination with other groups, means straight and branched divalent chain groups with one to seven carbon atoms, preferably one to four carbon atoms that can be optionally substituted by halogens. Examples of lower alkylene are methylene, ethylene, propylene or butylene.

The term lower alkenylene, alone or in combination with other groups, means straight and branched divalent chain groups comprising an olefinic bond and two to seven carbon atoms, preferably two to four carbon atoms, that can be optionally substituted by halogens. Examples of lower alkenylene are vinylene, propenylene and butenylene.

The term lower alkylenedioxy refers to a lower alkylene substituted at each end by an oxygen atom. Examples of lower alkylenedioxy groups are preferably methylenedioxy and ethylenedioxy.

The term lower alkylenoxy refers to a lower alkylene substituted at one end by an oxygen atom. Examples of lower alkylenoxy groups are preferably methylenoxy, ethylenoxy and propylenoxy.

The term halogen means fluorine, chlorine, bromine or iodine, preferably fluorine, chlorine and bromine.

The term cycloalkyl alone or in combination, means a saturated cyclic hydrocarbon ring system with 3 to 7 carbon atoms, e.g. cyclopropyl, cyclobutyl, cyclopentyl, cyclohexyl and cycloheptyl, which can be optionally mono-, di-, or trisubstituted independently by lower alkyl, lower alkenyl, lower alkenylene, lower alkoxy, lower alkylenoxy, lower alkylenedioxy, hydroxy, halogen, —CF₃, —NR¹R², —NR¹C(O)R², —NR¹S(O)₂R², —C(O)NR¹R², lower alkylcarbonyl, —COOR¹, —SR¹, —SOR¹, —SO₂R¹, —SO₂NR¹R². The cyclopropyl group is a preferred group, whereby R¹ and R² have the meaning given in formula (I) below, another aryl, another heteroaryl or another heterocyclyl and the like.

The term aryl, alone or in combination, relates to the phenyl, the naphthyl or the indanyl group, preferably the phenyl group, which can be optionally mono-, di-, tri-, tetra- or penta-substituted independently by lower alkyl, lower alkenyl, lower alkinyl, lower alkenylene or lower alkylene forming with the aryl ring a five- or six-membered ring, lower alkoxy, lower alkylenedioxy, lower alkylenoxy, hydroxy, hydroxy-lower alkyl, halogen, cyano, —CF₃, —OCF₃, —NR¹R², -lower alkyl —NR¹R², —NR¹C(O)R², —NR₁S(O)₂R², —C(O)NR¹R², —NO₂, lower alkylcarbonyl, —COOR¹, —SR¹, —S(O)R¹, —S(O)₂R¹, —SO₂NR¹R², benzyloxy. Preferred substituents are halogen, lower alkoxy, and lower alkyl. The substituents R¹ and R² have the meaning given in Formula (I) below.

For the substituent U, the term aryl, means for example a phenyl group which is mono-, di-, tri-, tetra- or penta-substituted independently by fluorine or chlorine, such as for example: 2-chloro-3,6-difluoro-phenyl.

For the substituent M, the term aryl, means for example a phenyl group which is mono-, di-, tri-, tetra- or pentasubstituted independently by fluorine or chlorine, such as for example: 2,3-dichloro-phenyl.

The term aryloxy refers to an Ar—O group, wherein Ar is an aryl. An example of aryloxy groups is phenoxy.

The term heterocyclyl, alone or in combination, means saturated or unsaturated (but not aromatic) five-, six- or seven-membered rings containing one or two nitrogen, oxygen or sulfur atoms which may be the same or different and which rings can be optionally substituted with lower alkyl, hydroxy, lower alkoxy and halogen. The nitrogen atoms, if present, can be substituted by a —COOR² group, whereby R² has the meaning given in the Formula (I) below. Examples of such rings are piperidinyl, morpholinyl, thiomorpholinyl, piperazinyl, tetrahydropyranyl, dihydropyranyl, 1,4-dioxanyl, pyrrolidinyl, tetrahydrofuranyl, dihydropyrrolyl, imidazolidinyl, dihydropyrazolyl, dihydroquinolinyl, tetrahydroquinolinyl, tetrahydroisoquinolinyl.

The term heteroaryl, alone or in combination, means six-membered aromatic rings containing one to four nitrogen atoms; benzofused six-membered aromatic rings containing one to three nitrogen atoms; five-membered aromatic rings containing one oxygen, one nitrogen or one sulfur atom; benzofused five-membered aromatic rings containing one oxygen, one nitrogen or one sulfur atom; five-membered aromatic rings containing one oxygen and one nitrogen atom and benzofused derivatives thereof; five-membered aromatic rings containing a sulfur and a nitrogen or an oxygen atom and benzofused derivatives thereof; five-membered aromatic rings containing two nitrogen atoms and benzofused derivatives thereof; five-membered aromatic rings containing three nitrogen atoms and benzofused derivatives thereof, or a tetrazolyl ring. Examples of such ring systems are furanyl, thiophenyl, pyrrolyl, pyridinyl, pyrimidinyl, indolyl, quinolinyl, isoquinolinyl, imidazolyl, triazinyl, thiazinyl, thiazolyl, isothiazolyl, pyridazinyl, pyrazolyl, oxazolyl, isoxazolyl, coumarinyl, benzothiophenyl, quinazolinyl, quinoxalinyl. Such rings may be adequately substituted with lower alkyl, lower alkenyl, lower alkinyl, lower alkylene, lower alkenylene, lower alkylenedioxy, lower alkyleneoxy, hydroxy-lower alkyl, lower alkoxy, hydroxy, halogen, cyano, —CF₃, —OCF₃, —NR¹R², —NR¹R²— lower alkyl, —N(R¹)COR¹, —N(R¹)SO₂R¹, —CONR¹R², —NO₂, lower alkylcarbonyl, —COOR¹, —SR¹, —S(O)R¹, —S(O)₂R¹, —SO₂NR¹R², whereby R¹ and R² have the meaning given in formula (I) below, another aryl, another heteroaryl or another heterocyclyl and the like. In another embodiment, and in addition to the above-mentioned substituents, the heteroaryl may additionally be substituted with a group hydroxyl-lower alkylene-oxy, wherein lower alkylene is as defined above (preferred example for lower alkylene is ethylene).

For the substituent W the term heteroaryl means for example pyridinyl thiazoyl, oxazoyl, and isoxazoyl. For the substituent U, the term heteroaryl means for example isoxazoyl, pyrrazoyl. For the substituent M, the term heteroaryl means for example pyridinyl substituted with lower alkyl, hydroxyl-lower alkylene-oxy, and lower alkoxy, such as 2-methoxy-3-methylpyridin-4-yl. A preferred example is 2-(3-hydroxypropoxy)-3-methylpyridin-4-ylmethyl.

The term heteroaryloxy refers to a Het-O group, wherein Het is a heteroaryl.

The term heteroaryl-lower alkyl means that a heteroaryl as define above is attached to a lower alkyl group as defined above. An example is pyridinyl-methyl. Further examples are the following heteroaryl groups attached to a methyl group: furanyl, thiophenyl, pyrrolyl, pyrimidinyl, indolyl, quinolinyl, isoquinolinyl, imidazolyl, triazinyl, thiazinyl, thiazolyl, isothiazolyl, pyridazinyl, pyrazolyl, oxazolyl, isoxazolyl, coumarinyl, benzothiophenyl, quinazolinyl and quinoxalinyl.

The term aryl-lower alkyl means that an aryl as define above is attached to a lower alkyl group as defined above. An example is phenyl-methyl (benzyl). Further examples are the following aryl groups attached to a methyl group: naphthyl and indanyl.

The term cycloalkyl-lower alkyl means that a cycloalkyl as define above is attached to a lower alkyl group as defined above. An example is cyclopropyl-methyl. Further examples are the following cycloalkyl groups attached to a methyl group: cyclobutyl, cyclopentyl, cyclohexyl and cycloheptyl.

It is understood that the substituents outlined relative to the expressions cycloalkyl, heterocyclyl, heteroaryl and aryl have been omitted in the definitions of the general formula (I) and in claims 1 to 5 for clarity reasons but the definitions in formula (I) and in claims 1 to 5 should be read as if they are included therein.

The expression pharmaceutically acceptable salts encompasses either salts with inorganic acids or organic acids like hydrochloric or hydrobromic acid, sulfuric acid, phosphoric acid, citric acid, formic acid, acetic acid, maleic acid, tartaric acid, benzoic acid, methanesulfonic acid, p-toluenesulfonic acid, and the like that are non toxic to living organisms or in case the compound of formula (I) is acidic in nature with an inorganic base like an alkali or earth alkali base, e.g. sodium hydroxide, potassium hydroxide, calcium hydroxide and the like.

The compounds of the general formula (I) can contain one or more asymmetric carbon atoms and may be prepared in form of optically pure enantiomers, mixtures of enantiomers such as racemates, diastereomers, mixtures of diastereomers, diastereomeric racemates, mixtures of diastereomeric racemates, and the meso-form and pharmaceutically acceptable salts thereof.

The present invention encompasses all these forms. Mixtures may be separated in a manner known per se, i.e. by column chromatography, thin layer chromatography, HPLC or crystallization.

A first aspect of the invention consists in novel tetrahydropyridine derivatives of the general formula (I).

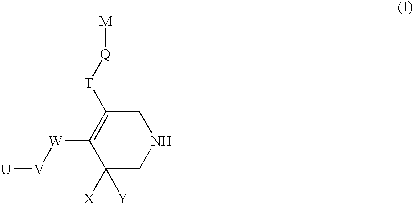

wherein
X and Y represent independently hydrogen, fluorine or a methyl group; X and Y do not represent both hydrogen at the same time or X and Y may together form a cyclopropyl ring;
W represents a phenyl or heteroaryl ring, the heteroaryl ring being a six-membered and non-fused ring, the phenyl ring and the heteroaryl ring are substituted with V in position 3 or 4;
V represents —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; —CH₂—CH₂-A-CH₂—CH₂—B—; —O—CH₂—CH(OCH₃)—CH₂—O; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)—CH₂—O—; —O—CH₂—C(CH₂CH₂)—O— or —O—C(CH₂CH₂)—CH₂—O—;
A and B independently represent —O—; —S—; —SO— or —SO₂—;
U represents aryl or heteroaryl;
T represents —CONR¹—; —(CH₂)ₚOCO—; —(CH₂)ₚN(R¹)CO—; —(CH₂)ₚN(R¹)SO₂—; —COO—; —(CH₂)ₚOCONR¹— or —(CH₂)ₚN(R²)CONR¹—;
R¹ and R² independently represent hydrogen; lower alkyl; lower alkenyl; lower alkinyl; cycloalkyl; aryl-lower alkyl, heteroaryl-lower alkyl or cycloalkyl-lower alkyl;
Q represents lower alkylene or lower alkenylene;
M represents hydrogen; cycloalkyl; aryl; heterocyclyl or heteroaryl;
p is the integer 1, 2, 3 or 4;
r is the integer 3, 4, 5, or 6;
s is the integer 2, 3, 4 or 5;
t is the integer 1, 2, 3 or 4;
u is the integer 1, 2 or 3;
v is the integer 2, 3 or 4.

In a further embodiment of the invention, the tetrahydropyridine derivatives of the general formula (I) as described above also encompass optically pure enantiomers, mixtures of enantiomers such as racemates, diastereomers, mixtures of diastereomers, diastereomeric racemates, mixtures of diastereomeric racemates, and the meso-form; as well as pharmaceutically acceptable salts, solvent complexes and morphological forms.

A group of preferred compounds of general formula (I) are those wherein X, Y, V, W and U are as defined in general formula (I) and wherein T represents —CONR¹—; Q represents a lower alkylene; M represents hydrogen, aryl or heteroaryl.

Another group of preferred compounds of general formula (I) are those wherein X, Y, W, T, Q and M are as defined in general formula (I), V represents —CH₂CH₂O—; —CH₂CH₂CH₂O—; —OCH₂CH₂O— or —CH₂CH₂CH₂OCH₂O— and U is as above-defined in general formula (I).

A group of more preferred compounds of general formula (I) are those wherein X, Y, V, U, T, Q and M are as defined in general formula (I) and W represents a phenyl substituted in 4-position with V.

Another group of particularly more preferred compounds of general formula (I) are those wherein W, V, U, T, Q, and M are as defined in general formula (I) and X and Y together may form a cyclopropyl group.

In another embodiment of the invention A and B independently represent —O—.

In another embodiment of the invention R¹ and R² independently represent cycloalkyl, such as cyclopropyl.

The group V as defined above such as -A-(CH₂)ₛ— is integrated into a compound of formula I in that A is attached to U and the alkylene part of -A-(CH₂)ₛ— is attached to W.

In a preferred embodiment p represents the integer 1.

In a preferred embodiment r represents the integer 3 or 4.

In a preferred embodiment s represents the integer 2 or 3.

In a preferred embodiment t represents the integer 1 or 2.

In a preferred embodiment u represents the integer 1 or 2.

In a preferred embodiment v represents the integer to 2 or 3. In a further preferred embodiment v represents the integer 2.

Most preferred compounds of general formula (I) are those selected from the group consisting of:
8-{4-[3-(2-Chloro-3,6-difluorophenoxy)propyl]phenyl}-5-aza-spiro[2.5]oct-7-ene-7-carboxylic acid cyclopropyl-(2,3-dichlorobenzyl)amide;
4-{4-[3-(2-Chloro-3,6-difluorophenoxy)propyl]phenyl}-5,5-dimethyl-1,2,5,6-tetrahydropyridine-3-carboxylic acid cyclopropyl-(2,3-dichlorobenzyl)amide;
4-{4-[3-(2-Chloro-3,6-difluorophenoxy)propyl]phenyl}-5,5-dimethyl-1,2,5,6-tetrahydro-pyridine-3-carboxylic acid cyclopropyl-(2-methoxy-3-methylpyridin-4-ylmethyl)amide;
8-{4-[3-(2-Chloro-3,6-difluorophenoxy)propyl]phenyl}-5-aza-spiro[2.5]oct-7-ene-7-carboxylic acid cyclopropyl-(2-methoxy-3-methylpyridin-4-yl-methyl)-amide;
8-{4-[3-(2-chloro-3,6-difluorophenoxy)propyl]phenyl}-5-azaspiro[2.5]oct-7-ene-7-carboxylic acid cyclopropyl-[2-(2-hydroxypropoxy)-3-methylpyridin-4-ylmethyl]amide;
4-{4-[3-(2-chloro-3,6-difluorophenoxy)propyl]phenyl}-5,5-dimethyl-1,2,5,6-tetrahydro-pyridine-3-carboxylic acid cyclopropyl-[2-(2-hydroxypropoxy)-3-methylpyridin-4-ylmethyl]amide;
4-{4-[3-(2-chloro-3,6-difluorophenoxy)propyl]phenyl}-5,5-difluoro-1,2,5,6-tetrahydropyridine-3-carboxylic acid cyclopropyl-(2,3-dichlorobenzyl)amide.

The invention relates to a method for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, which method comprises administrating a compound as defined above to a human being or animal.

In another embodiment, the invention relates to a method for the treatment and/or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, complications resulting from diabetes such as nephropathy, vasculopathy and neuropathy.

In another embodiment, the invention relates to a method for the treatment and/or prophylaxis of diseases, which are associated with a dysregulation of the renin-angiotensin system as well as for the treatment of the above-mentioned diseases.

The invention also relates to the use of compounds of formula (I) for the preparation of a medicament for the treatment and/or prophylaxis of the above-mentioned diseases.

A further aspect of the present invention is related to a pharmaceutical composition containing at least one compound according to general formula (I) and pharmaceutically acceptable carrier materials or adjuvants. This pharmaceutical composition may be used for the treatment or prophylaxis of the above-mentioned disorders; as well as for the preparation of a medicament for the treatment and/or prophylaxis of the above-mentioned diseases.

Derivatives of formula (I) or the above-mentioned pharmaceutical compositions are also of use in combination with other pharmacologically active compounds comprising ACE-inhibitors, neutral endopeptidase inhibitors, angiotensin II receptor antagonists, endothelin receptors antagonists, vasodilators, calcium antagonists, potassium activators, diuretics, sympatholitics, beta-adrenergic antagonists, alpha-adrenergic antagonists or with other drugs beneficial for the prevention or the treatment of the above-mentioned diseases.

In a preferred embodiment, this amount is comprised between 2 mg and 1000 mg per day.

In a particular preferred embodiment, this amount is comprised between 1 mg and 500 mg per day.

In a more particularly preferred embodiment, this amount is comprised between 5 mg and 200 mg per day.

All forms of prodrugs leading to an active component comprised by general formula (I) above are included in the present invention.

Compounds of formula (I) and their pharmaceutically acceptable acid addition salts can be used as medicaments, e.g. in the form of pharmaceutical compositions containing at least one compound of formula (I) and pharmaceutically acceptable inert carrier material or adjuvants. These pharmaceutical compositions can be used for enteral, parenteral, or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or infusion solutions, or topically, e.g. in the form of ointments, creams or oils.

The production of pharmaceutical preparations can be effected in a manner which will be familiar to any person skilled in the art by bringing the described compounds of formula (I) and their pharmaceutically acceptable acid addition salts, optionally in combination with other therapeutically valuable substances, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, cornstarch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers are, however, required in the case of soft gelatine capsules). Suitable carrier materials for the production of solutions and syrups are, for example, water, polyols, sucrose, invert sugar and the like. Suitable carrier materials for injections are, for example, water, alcohols, polyols, glycerols and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavor-improving agents, salts for varying the osmotic pressure, buffer substances, solubilizers, colorants and masking agents and antioxidants come into consideration as pharmaceutical adjuvants.

The dosage of compounds of formula (I) can vary within wide limits depending on the disease to be controlled, the age and the individual condition of the patient and the mode of administration, and will, of course, be fitted to the individual requirements in each particular case.

Another aspect of the invention is related to a process for the preparation of a pharmaceutical composition comprising a derivative of the general formula (I). According to said process, one or more active ingredients of the general formula (I) are mixing with inert excipients in a manner known per se.

The compounds of general formula I can be manufactured by the methods outlined below, by the methods described in the examples or by analogous methods.

The compounds of general formula (I) can be manufactured by the methods given below, by the methods given in the examples or by analogous methods. The tetrahydropyridine derivatives exemplified in this invention may be prepared from readily available starting materials using the following general methods and procedures. Optimum reaction conditions may vary with the particular reactants or solvents used, but such conditions can be determined by one skilled in the art by routine optimization procedures.

### Preparation of the Precursors:

Precursors are compounds, which were prepared as key intermediates and/or building blocks and are suitable for further transformations in parallel chemistry.

For instance, a compound of type A is prepared from a known 4-oxopiperidine derivative (Scheme 1), whereas PG represents a suitable protecting group. Subsequent acylation leads to a compound of type B (Majewski, M; et al.; J. Org. Chem., 1995, 60, 5825), whereas R^{a} is a suitable ester (e.g. ethyl, methyl and benzyl).

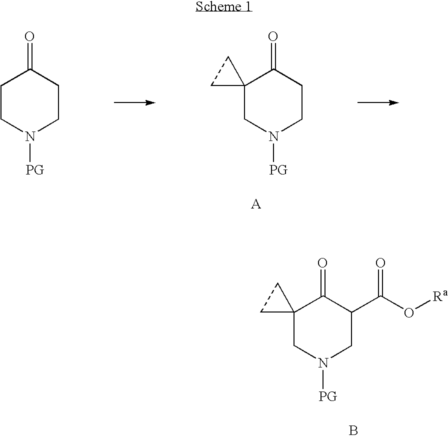

Compounds of formula B substituted with one or two methyl groups instead of a cyclopropyl group at the piperidinyl 5-position can be prepared by a similar methodology from known starting materials or following known literature (Patent application WO2001000577). Formation of the vinyl triflate C, followed by a coupling catalyzed by a Pd(0) complex leads to tetrahydropyridine derivatives of type D, wherein R^{b} optionally represents any U-V group as defined in general formula (I) or a chemical precursor of such a group (Scheme 2).

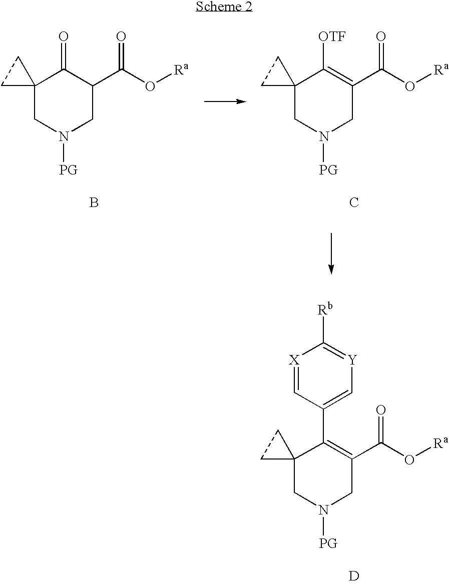

A compound of type D can be transprotected to a compound of type E, then coupled to a phenol or aromatic alcohol using a Mitsunobu reaction, leading to derivatives of type F wherein V and U have the meaning given in general formula I as above-mentioned. The ester F is optionally cleaved by any suitable method to lead to precursor G (Scheme 3).

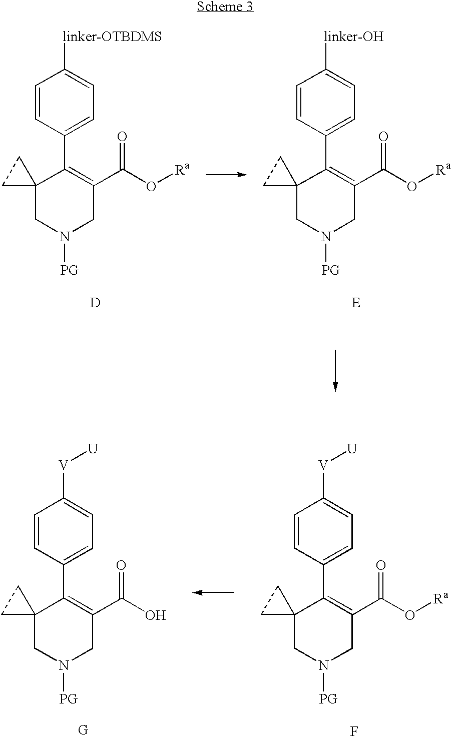

Also, a compound of type D can be reduced to a compound of type M that can be then oxidized to a compound of type N (Scheme 4). Aldehyde N can then be transformed to a compound of type O by reductive amination, which can be acylated to a derivative of type Q′ wherein Q and M have the meaning given in general formula (I) above. On the other hand, compounds of type M can be then acylated following standard procedures to esters or carbamates of type P.

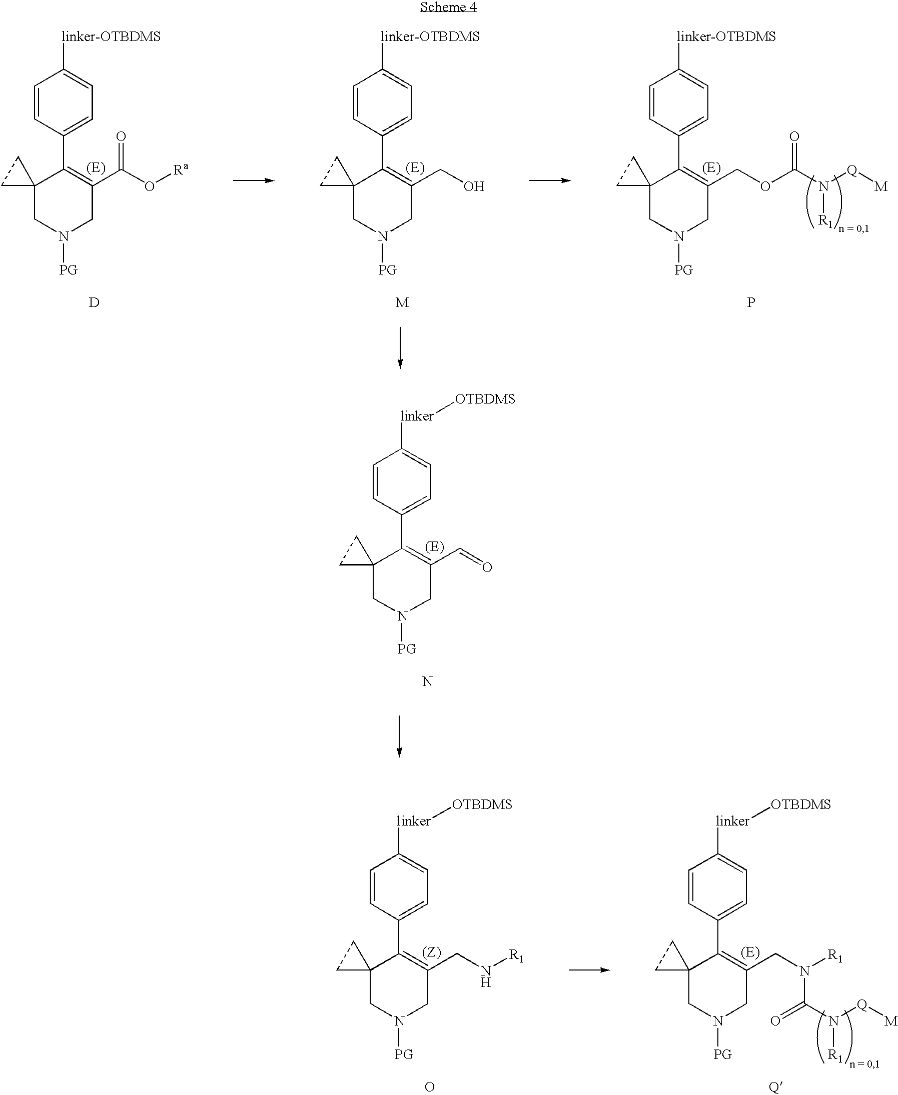

Preparation of a monofluorated derivative can start from the commercially available N-protected piperidin-4-one S (Scheme 5). Fluorination by a reagent delivering an F⁺-synthon, like DAST or Selectfluor®, can lead to a derivatives of type S′. Acylation with nitriloacetic acid methyl ester for instance can lead to derivatives of type T′. Then a similar chemistry can be used as described here above (Schemes 2-4).

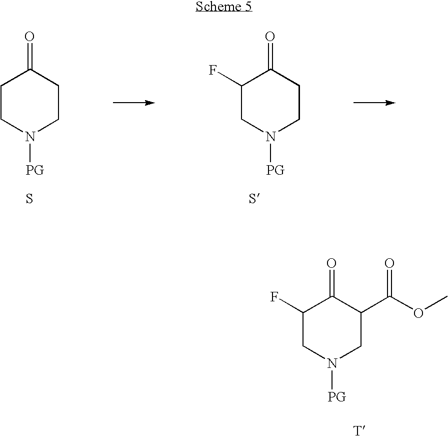

A difluorinated derivative of type T″ must be prepared through a different way (Scheme 6). Condensation of N-benzyl-β-alanine ethyl ester with formaldehyde and benzotriazol yields compound V. Compound W is obtained following a reaction with a Reformasky type reagent. Then a Dieckmann cyclization leads to compound T″, which is structurally similar to compound C (Scheme 2).

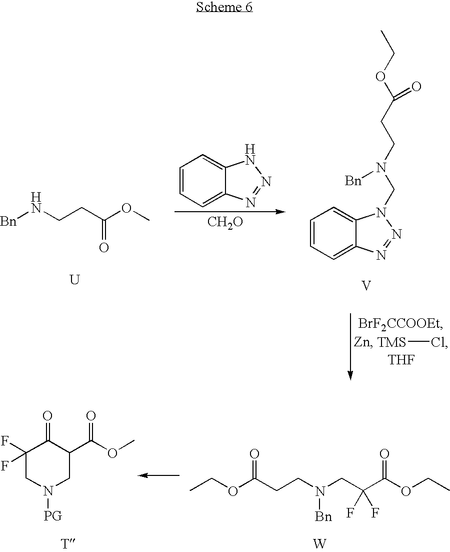

### Preparation of Bromoaryl Derivatives

For the coupling of compounds of type C to tetrahydropyridine derivatives of type D (cf. Scheme 2), it may be necessary to prepare the bromoaryl components needed as described in Scheme 7. A Mitsunobu coupling leading to compounds of type H, or the alkylation of an alcohol with a benzylic chloride (or bromide) leading to compounds of type J are often the most convenient methods. Derivative K was prepared in one step from 1-(3-chloropropoxymethyl)-2-methoxybenzene by reaction with 4-bromophenol (Vieira E. et al., Bioorg. Med. Chem. Letters, 1999, 9, 1397). Other methods for the preparation of ethers or thioethers, like a Williamson synthesis, can be used as well (see e.g. March, J, “Advanced Organic Chemistry”, 5^{th} ed., John Wiley and sons, 2001).

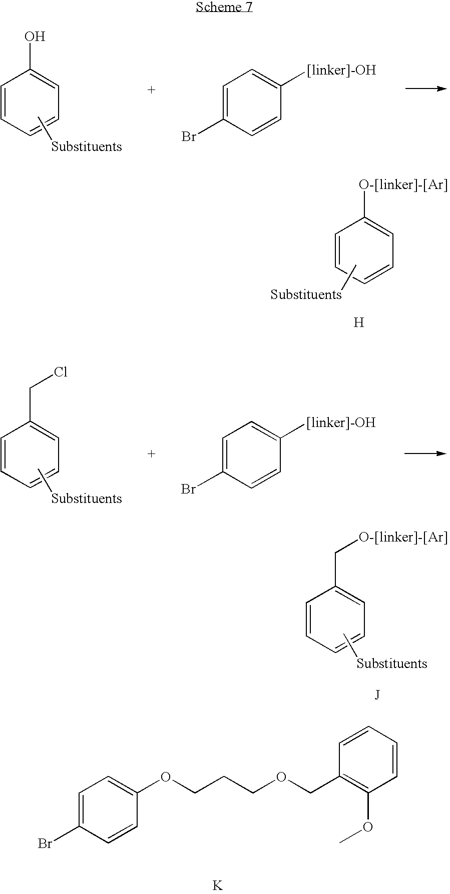

### Preparation of Final Compounds

A compound of type G can be coupled to the amine to yield amides of type L wherein V, U and M have the meaning given in general formula (I) above. Removal of the N-protecting group (PG) leads to the final compounds of type R wherein V, U, Q and M have the meaning given in general formula (I) above (Scheme 8).

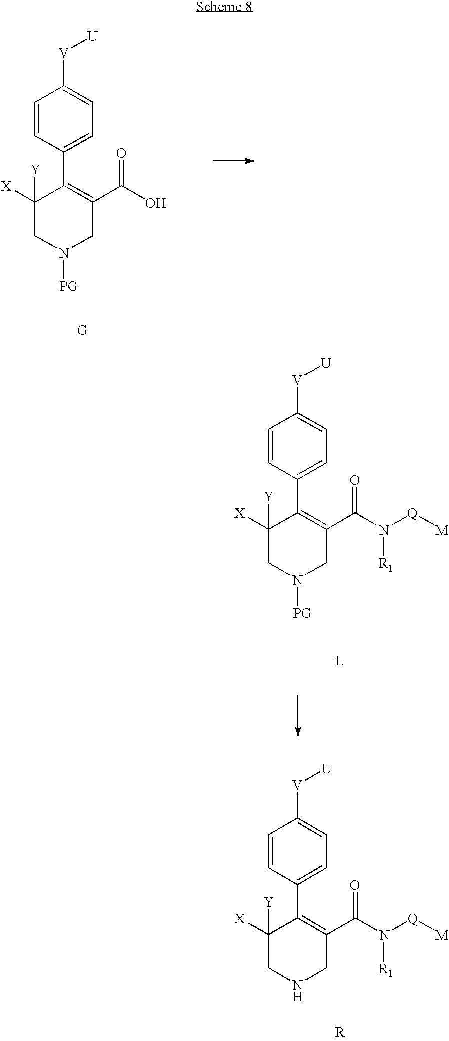

Also, compounds of type P or Q′ (Scheme 4) may be processed further as indicated in Scheme 3, then deprotected as indicated in Scheme 8, to lead to final compounds as defined in general formula (I) The two following general procedures for amide coupling and removal of a Boc-protecting group are used.

### General Remarks

The following compounds were prepared according to the procedures described for the synthesis of compounds encompassed by the general formula (I). All compounds were characterized by ¹H-NMR (300 MHz) and occasionally by ¹³C-NMR (75 MHz) (Varian Oxford, 300 MHz; chemical shifts are given in ppm relative to TMS), by LC-MS: A: 2 min<t_{R}<10 min; (Waters Micromass; ZMD-platform with ESI-probe with Alliance 2790 HT; Column: 2×30 mm, Gromsil ODS4, 3 μM, 120A; Gradient: 0-100% acetonitrile in water, 6 min, with 0.05% formic acid, flow: 0.45 mL/min; t_{R} given in min.), B: 0.1 min<t_{R}<2 min; (Finnigan AQA with ESI-probe with HP 110 DAD and HP110 binary pump; column: Develosil RP-AQUEOUS, 5 μM, 4.6 mm×50 mm; gradient: 5-95% methanol in water (0.04% TFA), 1 min, 95% methanol in water (0.04% TFA) 0.4 min, 4.5 mL/min.), by TLC (TLC-plates from Merck, Silica gel 60 F₂₅₄).

### Abbreviations

### ACE Angiotensin Converting Enzyme

### Ang Angiotensin

aq. aqueous
9-BBN 9-Borabicyclo[3.3.1]nonane

### Bn Benzyl

Boc tert-Butyloxycarbonyl
BSA Bovine serum albumine
BuLi n-Butyllithium
conc. Concentrated
DAST Diethylaminosulfur trifluoride
DIBAL Diisobutylaluminium hydride

### DIPEA Diisopropylethylamine

### DMAP 4-N,N-Dimethylaminopyridine

### DMF N,N-Dimethylformamide

### DMSO Dimethylsulfoxide

EDC.HCl Ethyl-N,N-dimethylaminopropylcarbodiimide hydrochloride
EIA Enzyme immunoassay
eq. equivalent

### Et Ethyl

EtOAc Ethyl acetate

### FC Flash Chromatography

### HOBt Hydroxybenzotriazol

### MeOH Methanol

org. organic

### PBS Phosphate Buffer Saline

PG protecting group

### Ph Phenyl

### RAS Renin Angiotensin System

RP18 Reversed phase column, filled with C₁₈ hydrocarbon
rt room temperature

### Selectfluor®

sol. Solution
TBAF Tetrabutylammonium fluoride
TBDMS tert-Butyldimethylsilyl
^{t}BuOH tert-Butanol
^{t}BuOK Potassium tert-butylate

### Tf Trifluoromethylsulfonyl

TFA Trifluoroacetic acid

### THF Tetrahydrofuran

### TLC Thin Layer Chromatography

### TMAD N,N,N′,N′-Tetramethylazodicarboxamide

### General Procedures

### General Procedure A

A sol. of the desired carboxylic acid (1.00 eq), the desired amine (2.00 eq), EDC.HCl (1.10 eq.), HOBt (cat. amount), DMAP (cat. amount) and DIPEA (2.00 eq.) in CH₂Cl₂ (20 mL/g of acid) was stirred at rt overnight. The reaction mixture was washed over diatomic earth (Isolute Sorbent Technology, Johnson, C. R., et al., Tetrahedron, 1998, 54, 4097) and the org. extracts were evaporated under reduced pressure. The residue was used without further purification.

### General Procedure B

The starting material was dissolved in CH₂Cl₂ (10 mL/g of starting material) and the sol. was cooled to 0° C. 4M HCl in dioxane (same volume as CH₂Cl₂) was added and the reaction mixture was left for 90 min at rt. The solvents were removed under reduced pressure. Purification of the residue by HPLC led to the desired compound.

### {2-[2-(tert-Butyldimethylsilanyloxy)propoxy]-3-methylpyridin-4-ylmethyl}cyclopropyl-amine

2-Chloro-N-phenylisonicotinamide: To the sol. of 2-chloroisonicotinoyl chloride (Anderson, W. K., Dean, D. C., Endo, T., J. Med. Chem., 1990, 33, 1667, 10 g, 56.8 mmol) in 1,2-dichloroethane (100 mL) was added at 0° C. a sol. of aniline (5.70 mL, 62.5 mmol) and DIPEA (10.2 ml, 59.6 mmol) in 1,2-dichloroethane (10 ml) during ca. 30 min. The reaction was stirred at 0° C. for ca. 30 min and subsequently for 1 h at 95° C. Water (30 mL) was added at rt and the mixture was filtered-off. The filtrate was extracted with CH₂Cl₂ (200 mL). The combined org. extracts were dried over MgSO₄, filtered, and the solvents were removed under reduced pressure. The residue was crystallized from MeOH/water 1:10 (110 mL), yielding the title compound (12.12 g, 92%). LC-MS: R_{T}=0.87 min; ES⁺=233.1. 2-Chloro-3-N-dimethyl-N-phenylisonicotinamide: To a sol. of compound N (8.79 g, 37.8 mmol) in THF (90 mL) was added BuLi (1.6M in hexane, 52 mL, 83.2 mmol) at −78° C. After 30 min MeI (7.70 mL, 124 mmol) was added dropwise at the same temperature. The mixture was stirred at −78° C. for 1 h, and was warmed up to 33° C. The mixture was stirred at 33° C. for 30 min. Aq. 10% NH₄OH was added dropwise at rt, and the mixture was extracted with Et₂O. The org. extracts were dried over MgSO₄, filtered, and the solvents were evaporated under reduced pressure. Purification by FC yielded the title compound (8.67 g, 88%). LC-MS:R_{T}=0.85 min; ES⁺=261.2. 2-Chloro-3-methylpyridine-4-carbaldehyde: To the sol. of pyridine derivative O (9.58 g, 36.7 mmol) in CH₂Cl₂ (190 mL) was at −78° C. added DIBAL (1M in CH₂Cl₂, 55.1 mL, 55.1 mmol), and the mixture was stirred at −78° C. for 1.5 h. Aq. sat. tartaric acid monosodium monokalium salt in water (20 ml) was added and the mixture was allowed to warm up to rt. Water was added and the mixture was extracted with CH₂Cl₂. The org. extracts were dried over MgSO₄, filtered, and the solvents were removed under reduced pressure. Purification of the residue by FC yielded the title compound (4.4 g, 77%). LC-MS:R_{T}=0.76 min; ES⁺=156.1. (2-Chloro-3-methylpyridin-4-ylmethyl)-cyclopropylamine: A sol. of aldehyde P (4.70 g, 30.2 mmol) and cyclopropylamine (4.20 ml, 60.4 mmol) in MeOH (65 mL) was stirred at rt for 4 h. NaBH₄ (1.55 g, 39.2 mmol) was added and the mixture was stirred at rt for 12 h. Water and subsequently aq. 1M NaOH were added, and the solvents were partially removed under reduced pressure. The water phase was extracted with CH₂Cl₂ (2×). The combined org. extracts were dried over MgSO₄, filtered, and the solvents were removed under reduced pressure. Purification of the crude by FC yielded the title compound (4.66 g, 79%). LC-MS:R_{T}=0.43 min; ES⁺=197.1. {2-[2-(tert-Butyldimethylsilanyloxy)propoxy]-3-methylpyridin-4-ylmethyl}cyclopropylamine: A sol. of amine Q (1.30 g, 6.61 mmol) and 2-(tert-butyldimethylsilanyloxy)propanol (433 mg, 10.58 mmol) in dioxane (5 ml) was heated at 115° C. for 12 h. The solvents were removed under reduced pressure, water was added, and the mixture was extracted with Et₂O (2×). The combined org. extracts were dried over MgSO₄, filtered, and the solvents were removed under reduced pressure. Purification of the crude by FC yielded the title compound (926 mg, 42%). LC-MS:R_{T}=0.79 min; ES⁺=337.3.

### Precursors

### 8-Oxo-5-azaspiro[2.5]octane-5-carboxylic acid tert-butyl ester (A1)

To a sol. of ^{t}BuOK (0.28 g, 2.5 mmol) in ^{t}BuOH (4 mL) was added 1-Boc-4-piperidone (0.50 g, 2.5 mmol). After 5 min of stirring, 2-chloroethyldimethyl sulfonium iodide (0.57 g, 2.25 mmol, P. Kraft, Synthesis, 1999, 4, 695) was added in portions over 15 min. After 2 h stirring, a sol. of ^{t}BuOK (0.28 g, 2.5 mmol) in ^{t}BuOH (4 mL) was added again and stirring was continued overnight. The reaction mixture was poured into water and extracted with EtOAc (3×). The combined organic phases were dried over Na₂SO₄, filtered, and the solvents were removed under reduced pressure. Purification of the residue by FC (EA/heptane, 1/9, 3/7, 1/1) yielded the title compound (0.22 g, 40%).

### 3,3-Dimethyl-4-oxopiperidine-1-carboxylic acid tert-butyl ester (A2)

NaH (60% susp. in oil, 5.71 g, 143 mmol) was added to a sol. of N-Boc-4-piperidone (13.6 g, 68.0 mmol) in THF (350 mL) at 0° C. MeI (10.6 mL, 170 mmol) was added. The mixture was stirred at 0° C. for 30 min, and was allowed to warm to rt. Sat. aq. NH₄Cl was added, and the mixture was extracted with EtOAc. The org. extracts were washed with brine, dried over MgSO₄, filtered, and the solvents were removed under reduced pressure. Purification by FC (EtOAc/heptane 8:2) and then crystallization from heptane yielded the title compound (11.0 g, 73%).

### 8-Oxo-5-aza-spiro[2.5]octane-5,7-dicarboxylic acid 5-tert-butyl ester 7-methyl ester (B1)

To a sol. of diisopropylamine (1.4 mL, 9.9 mmol) in THF (50 mL) at −78° C. was added dropwise n-BuLi (1.6M in hexane, 6.6 mL, 9.9 mmol). The solution was stirred for 1 h at −78° C. A sol. of compound A1 (2.03 g, 9 mmol) in THF (20 mL) was added dropwise. The reaction mixture was stirred for 3 h at −78° C., and then methylcyanoformate (0.93 mL, 11.7 mmol) was added. The reaction mixture was stirred for 30 min at −78° C. and a sol. of AgNO₃ (2.2 g, 12.9 mmol) in H₂O/THF (1:1, 20 mL) was added. After 10 min H₂O (15 mL) and AcOH (15 mL) were added and the reaction mixture was allowed to warm to rt. Aq. 25% NH₃ was added until the Ag-salts had completely dissolved. The reaction mixture was extracted with EtOAc (1×) and CH₂Cl₂ (2×). The combined org. extracts were dried over MgSO₄, filtered, and the solvents were removed under reduced pressure. Purification of the residue by FC (EtOAc/heptane 1:9) yielded the title compound (1.32 g, 52%). LC-MS: t_{R}=1.03 min; ES⁺: 284.10.

### 5,5-Dimethyl-4-oxo-piperidine-1,3-dicarboxylic acid 1-tert-butyl ester 3-methyl ester (B2)

A sol. of diisopropylamine (4.20 mL, 29.7 mmol) in THF (90 mL) was cooled to −78° C. BuLi (1.6 M in hexane, 19.8 mL, 29.7 mmol) was added, and the sol. was stirred at −78° C. for 1 h. Compound A2 (6.14 g, 27 mmol) in THF (60 mL) was added, and the mixture was stirred for 3 h at −78° C. Methylcyanoformate (2.79 mL, 35.1 mmol) was added, and the mixture was stirred at −78° C. for 30 min. A sol. of AgNO₃ (6.56 g, 38.6 mmol) in H₂O/THF (1:1, 60 mL) was added. After 10 min., H₂O (45 mL) and AcOH (45 mL) were added and the reaction mixture was allowed to warm to rt. Ammoniac (25% in water) was added until the Ag-salt had completely dissolved. The reaction mixture was extracted with EtOAc (1×) and CH₂Cl₂ (2×). The combined org. extracts were dried over MgSO₄ and the solvents were removed under reduced pressure. Purification of the residue by FC (EtOAc/heptane 1:19→1:9) yielded the title compound (6.01 g, 78%). LC-MS: t_{R}=1.03 min.

### 8-Trifluoromethanesulfonyloxy-5-aza-spiro[2.5]oct-7-ene-5,7-dicarboxylic acid 5-tert-butyl ester 7-methyl ester (C1)

To a suspension of NaH (in oil, 55-65%, 0.72 g, about 18 mmol) in THF (60 mL) was added, at 0° C., a sol. of compound B1 (2.55 g, 9.00 mmol) in THF (20 mL). The suspension was stirred for 30 min at 0° C. Tf₂NPh (4.8 g, 13.5 mmol) was added at rt, and the reaction mixture was stirred for 18 h at 50° C. The mixture was allowed to cool to rt, ice was added, and the solvents were removed under reduced pressure. The residue was diluted with EtOAc and washed with aq. 10% Na₂CO₃. The org. extracts were dried over MgSO₄, filtered, and the solvents were removed under reduced pressure. Purification of the residue by FC (EtOAc/heptane 1:4) yielded the title compound (2.10 g, 56%). LC-MS: t_{R}=1.08 min; ES+: 416.03.

### 5,5-Dimethyl-4-trifluoromethanesulfonyloxy-5,6-dihydro-2H-pyridine-1,3-dicarboxylic acid 1-tert-butyl ester 3-methyl ester (C2)

Prepared in analogy to the preparation of compound C1, but from compound B2. Purification of the residue by FC (MeOH/CH₂Cl₂ 1:19→1:9) yielded the title compound (2.15 g, 80%). LC-MS: t_{R}=1.09 min.

### 1-Benzyl-5,5-difluoro-4-trifluoromethanesulfonyloxy-1,2,5,6-tetrahydro-pyridine-3-carboxylic acid ethyl ester (C3)

To a suspension of NaH (about 60% in oil, 0.27 g, about 6.8 mmol) in THF (20 mL) was added compound T″ (1.01 g, 3.4 mmol) in THF (15 mL), at 0° C. After 30 min the ice bath was removed and Tf₂NPh (1.82 g, 5.1 mmol) was added. The mixture was heated at 45° C. for 72 h. The mixture was allowed to cool to rt, ice was added, and the THF was evaporated under reduced pressure. EtOAc was added, the phases were separated and the organic phase was washed with aq. 10% Na₂CO₃ (1×). The org. extracts were dried over MgSO₄, filtered, and the solvents were removed under reduced pressure. Purification of the residue by FC (EtOAc/heptane 5:95→1:9) yielded the title compound (1.46 g, quantitative yield). LC-MS: Rₜ=1.13 min, ES+: 430.13.

### 8-{4-[3-(tert-butyldimethylsilanyloxy)propyl]phenyl}-5-aza-spiro[2.5]oct-7-ene-5,7-dicarboxylic acid 5-tert-butyl ester 7-methyl ester (D1)

To a sol. of [3-(4-bromophenyl)propoxy]-tert-butyldimethylsilane (Kiesewetter D. O., Tetrahedron Asymmetry, 1993, 4, 2183; 0.82 g, 2.5 mmol) in THF (10 mL) at −78° C. was added BuLi (1.5M in hexane, 1.7 mL, 2.56 mmol). The sol. was stirred at −78° C. for 30 min, and ZnCl₂ (1M in THF, 3 mL, 3 mmol) was added. The resulting sol. was allowed to warm to rt, and compound C1 (0.41 g, 1 mmol) and Pd(PPh₃)₄ (23 mg, 0.02 mmol) were added. After 20 min at rt ice was added to the reaction mixture. The solvents were removed under reduced pressure and the residue diluted with EtOAc. This mixture was washed with aq. 1M NaOH. The org. extracts were dried over MgSO₄, filtered, and the solvents were removed under reduced pressure. Purification of the residue by FC (EtOAc/heptane 1:9) led to the title compound (0.75 g, 56%). LC-MS: t_{R}=1.28 min; ES+: 516.42.

### 4-{4-[3-(tert-Butyldimethylsilanyloxy)propyl]phenyl}-5,5-dimethyl-5,6-dihydro-2H-pyridine-1,3-dicarboxylic acid 1-tert-butyl ester 3-methyl ester (D2)

Prepared in analogy to the preparation of compound D1, but from compound C2. Purification of the residue by FC (EtOAc/heptane 1:9) yielded the title compound (832 mg, 40%). LC-MS: t_{R}=1.29 min; ES+: 518.28.

### 1-Benzyl-4-{4-[3-(tert-butyldimethylsilanyloxy)propyl]phenyl}-5,5-difluoro-1,2,5,6-tetrahydropyridine-3-carboxylic acid ethyl ester (D3)

To a sol. of [3-(4-bromophenyl)propoxy]-tert-butyldimethylsilane (Kiesewetter D. O., Tetrahedron Asymmetry, 1993, 4, 2183; 1.69 g, 5.1 mmol) in THF (15 mL) at −78° C. was added BuLi (1.6M in hexane, 3.4 mL, 5.4 mmol). The sol. was stirred at −78° C. for 30 min, and ZnCl₂ (1M in THF, 5.78 mL, 5.78 mmol) was added. The resulting sol. was allowed to warm to rt, and compound C3 (1.46 g, 3.4 mmol) in THF (10 mL) and Pd(PPh₃)₄ (98 mg, 0.08 mmol) were added. The reaction mixture was heated at 45° C. for 18 h. Ice was added, the solvents were removed under reduced pressure, and the residue was diluted with EtOAc. This mixture was washed with aq. 1M NaOH. The org. phase was dried over MgSO₄, filtered, and the solvents were removed under reduced pressure. Purification of the residue by FC (EtOAc/heptane 5:95→1:9) led to the title compound (1.25 g, 69%). LC-MS: Rₜ=1.28 min, ES+=530.39.

### 8-[4-(3-Hydroxypropyl)phenyl]-5-aza-spiro[2.5]oct-7-ene-5,7-dicarboxylic acid 5-tert-butyl ester 7-methyl ester (E1)

TBAF (1.90 g, 6.00 mmol) was added to a sol. of compound D1 (0.94 g, 1.82 mmol) in THF (13 mL). The reaction mixture was stirred for 6 h at rt and diluted with EtOAc. The resulting mixture was washed with water and brine. The org. extracts were dried over MgSO₄, filtered and the solvents were removed under reduced pressure. Purification of the residue by FC (EtOAc/heptane 2:3) yielded the title compound (0.58 g, 80%). LC-MS: t_{R}=1.01 min; ES+: 402.21.

### 4-[4-(3-Hydroxypropyl)phenyl]-5,5-dimethyl-5,6-dihydro-2H-pyridine-1,3-dicarboxylic acid 1-tert-butyl ester 3-methyl ester (E2)

Prepared in analogy to the preparation of compound E1, but from compound D2. Purification of the residue by FC (EtOAc/heptane 1:1) yielded the title compound (0.41 g, 64%). LC-MS: t_{R}=1.02 min; ES+: 404.16, weak.

### 5,5-Difluoro-4-[4-(3-hydroxypropyl)phenyl]-5,6-dihydro-2H-pyridine-1,3-dicarboxylic acid 1-tert-butyl ester 3-ethyl ester (E3)

A solution of compound D3 (1.11 g, 2.1 mmol) and Boc₂O (0.5 g, 2.3 mmol) in EtOH (10 mL) was purged with N₂. Pd/C (10%, 0.1 g) was added and the suspension was purged with H₂. The reaction mixture was stirred under an H₂-atmosphere for 24 h and then filtered through Celite. The filtrate was evaporated under reduced pressure. Purification of the residue by FC (EtOAc/heptane 1:1) yielded the title compound (0.8 g, 90%). LC-MS: Rₜ=1.02 min, ES+=426.24.

### 8-{4-[3-(2-Chloro-3,6-difluorophenoxy)propyl]phenyl}-5-aza-spiro[2.5]oct-7-ene-5,7-dicarboxylic acid 5-tert-butyl ester 7-methyl ester (F1)

A sol. of compound E1 (580 mg, 1.45 mmol), 2-chloro-3,6-difluorophenol (280 mg, 1.74 mmol), azodicarboxyl dipiperidide (550 mg, 2.17 mmol) and tributylphosphine (0.71 mL, 2.9 mmol) in toluene (14 mL) was stirred for 1 h at rt, then for 1 h at 80° C. The reaction mixture was allowed to cool to rt, diluted with EtOAc and washed with water. The org. extracts were dried over MgSO₄, filtered, and the solvents were removed under reduced pressure. Purification of the residue by FC (EtOAc/heptane 1:19→1:9→1:4) led to the title compound (0.71 g, 89%). LC-MS: t_{R}=1.23 min; ES+: 548.23.

### 4-{4-[3-(2-Chloro-3,6-difluorophenoxy)propyl]phenyl}-5,5-dimethyl-5,6-dihydro-2H-pyridine-1,3-dicarboxylic acid 1-tert-butyl ester 3-methyl ester (F2)

Prepared in analogy to the preparation of compound F1, but from compound E2. Purification of the residue by FC (EtOAc/heptane 1:19→1:9→2:8) yielded the title compound (0.31 g, 57%). LC-MS: t_{R}=1.25 min.

### 4-{4-[3-(2-Chloro-3,6-difluorophenoxy)propyl]phenyl}-5,5-difluoro-5,6-dihydro-2H-pyridine-1,3-dicarboxylic acid 1-tert-butyl ester 3-ethyl ester (F3)

A sol. of compound E3 (0.79 g, 1.92 mmol), 2-chloro-3,6-difluorophenol (0.38 g, 2.30 mmol), azodicarboxyl dipiperidide (0.73 g, 2.88 mmol), and tributylphosphine (0.95 mL, 3.84 mmol) in toluene (20 mL) was stirred for 30 min at rt, then for 1 h at 65° C. The reaction mixture was allowed to cool to rt, was diluted with EtOAc and washed with water. The org. extract was dried over Na₂SO₄, filtered, and the solvents were removed under reduced pressure. Purification of the residue by FC (EtOAc/heptane 1:9→2:8) led to the title compound (1.1 g, quantitative yield). LC-MS: Rₜ=1.22 min, ES+=572.36, 516.24.

### 8-{4-[3-(2-Chloro-3,6-difluorophenoxy)propyl]phenyl}-5-aza-spiro[2.5]oct-7-ene-5,7-dicarboxylic acid 5-tert-butyl ester (G1)

To a sol. of compound F1 (712 mg, 1.30 mmol) in EtOH (13 mL) was added aq. 1M NaOH (13 mL). The resulting mixture was stirred for 90 min at 80° C., then allowed to cool to rt. Aq. 1M HCl (13 mL) was added, and the resulting mixture was extracted with EtOAc (3×). The combined org. extracts were dried over MgSO₄, filtered, and the solvents were removed under reduced pressure. Purification of the residue by FC (EtOAc/heptane 3:7→1:1) led to the title compound (0.70 g, quantitative yield). LC-MS: t_{R}=1.15 min; ES+: 534.16.

### 4-{4-[3-(2-Chloro-3,6-difluorophenoxy)propyl]phenyl}-5,5-dimethyl-5,6-dihydro-2H-pyridine-1,3-dicarboxylic acid 1-tert-butyl ester (G2)

Prepared in analogy to the preparation of compound G1, but from compound F2. Purification of the residue by FC (EtOAc/heptane 2:3→1:1) yielded the title compound (0.27 g, 89%). LC-MS: t_{R}=1.16 min.

### 4-{4-[3-(2-Chloro-3,6-difluorophenoxy)propyl]phenyl}-5,5-difluoro-5,6-dihydro-2H-pyridine-1,3-dicarboxylic acid 1-tert-butyl ester (G3)

To a sol. of compound F3 (1.09 g, 1.90 mmol) in EtOH (19 mL) was added aq. 1M NaOH (19 mL). The resulting mixture was stirred for 4 h at rt, then aq. 1M HCl (20 mL) was added and the resulting mixture was extracted with EtOAc (3×). The combined org. extracts were dried over MgSO₄, filtered and the solvents were removed under reduced pressure. The title compound was not further purified. LC-MS: Rₜ=1.22 min, ES+=544.18.

### 3-(Benzotriazol-1-ylmethylbenzylamino)propionic acid ethyl ester (V)

N-Benzyl-β-alanine ethyl ester (2.07 g, 10 mmol), followed by 37% aqueous formaldehyde (0.99 mL, 12 mmol) were added to a solution of benzotriazole (1.19 g, 10 mmol) in MeOH (7 mL). The solution was stirred overnight, and then the solvent was evaporated under reduced pressure. Purification of the residue by FC (EtOAc/heptane 3:7) yielded the title compound (3.24 g, 96%).

### 3-[Benzyl-(2-ethoxycarbonylethyl)amino]-2,2-difluoropropionic acid ethyl ester (W)

To a suspension of zinc dust (1.25 g, 19.2 mmol) in dry THF (15 mL), under nitrogen, was added TMS-Cl (1.27 ml, 10.1 mmol). After 10 min ethyl bromodifluoroacetate (1.36 mL, 10.6 mmol) was slowly added, followed 10 min later by a sol. of compound V (3.24 g, 9.6 mmol) in THF (6 mL). After, 18 h stirring at rt, the mixture was poured on 5% aqueous NaHCO₃ (20 mL) and filtered on Celite. The layers were separated and the aq. phase was extracted with EtOAc (2×20 mL). The combined org. layers were washed with 1M HCl (40 mL), and then dried over MgSO₄. After evaporation of the solvents under reduced pressure, the residue was diluted in ether. The formed solid was removed by filtration and the ether was evaporated. Purification of the residue by FC (EtOAc/heptane 2:8) yielded the title compound (3.07 g, 93%). LC-MS: Rₜ=1.06 min, ES+=344.26.

### 1-Benzyl-5,5-difluoro-4-hydroxy-1,2,5,6-tetrahydropyridine-3-carboxylic acid ethyl ester (T″)

To a solution of diisopropylamine (2.08 mL, 14.9 mmol) in THF (150 mL) under nitrogen and at −78° C., was added BuLi (1.6 M in hexane, 8.52 mL, 13.64 mmol), followed 45 min later by compound W (2.13 g, 6.2 mmol) in THF (50 mL). The cooling bath was removed and the reaction mixture warmed up slowly overnight. Sat. aq. NH₄Cl (200 mL) was added and the layers were separated. The aqueous phase was extracted with EtOAc (3×150 mL). The combined organic layers were washed with brine and dried over Na₂SO₄. Purification of the residue by FC (EtOAc/heptane 1:9) gave the title compound (1.5 g, 81%). LC-MS: Rₜ=1.04 min, ES+=298.22.

### EXAMPLES

### Example 1

### 8-{4-[3-(2-Chloro-3,6-difluorophenoxy)propyl]phenyl}-5-azaspiro[2.5]oct-7-ene-7-carboxylic acid cyclopropyl-[2-(2-hydroxypropoxy)-3-methylpyridin-4-ylmethyl]amide

According to general procedures A and B, starting from compound G1 (0.1 mmol) and {2-[2-(tert-butyldimethylsilanyloxy)propoxy]-3-methylpyridin-4-ylmethyl}cyclopropylamine.

### Example 2

### 4-{4-[3-(2-Chloro-3,6-difluorophenoxy)propyl]phenyl}-5,5-dimethyl-1,2,5,6-tetrahydro-pyridine-3-carboxylic acid cyclopropyl-[2-(2-hydroxypropoxy)-3-methylpyridin-4-ylmethyl]amide

According to general procedures A and B, starting from compound G2 (0.1 mmol) and {2-[2-(tert-butyldimethylsilanyloxy)propoxy]-3-methylpyridin-4-ylmethyl}cyclopropylamine. LC-MS: Rₜ=0.89 min, ES+: 654.32.

### Example 3

### 4-{4-[3-(2-Chloro-3,6-difluorophenoxy)propyl]phenyl}-5,5-difluoro-1,2,5,6-tetrahydropyridine-3-carboxylic acid cyclopropyl(2,3-dichlorobenzyl)amide

A sol. of compound G3 (0.52 g, 0.95 mmol), (2,3-dichlorobenzyl)cyclopropylamine (0.61 g, 2.85 mmol), DMAP (0.03 g, 0.24 mmol), DIPEA (0.66 mL, 3.8 mmol), HOBt (0.18 g, 1.19 mmol) and EDC.HCl (0.27 g, 1.42 mmol) in CH₂Cl₂ (20 mL) was stirred 72 h. The mixture was diluted with CH₂Cl₂, washed with aq. 1M HCl (2×) and brine (1×). The organic phase was dried over MgSO₄, filtered, and the solvents were removed under reduced pressure. Purification of the residue by FC (EtOAc/heptane 1:9→2:8→4:6) yielded the title compound (0.38 g, two steps: 54%). LC-MS: Rₜ=1.29 min, ES+=743.37. The former compound was dissolved in CH₂Cl₂ (5 mL) and the sol. was cooled to 0° C. 4M HCl in dioxane (5 mL) was added and the reaction mixture was stirred for 90 min at rt. The solvents were removed under reduced pressure. The residue was dissolved in EtOAc and washed with 1N NaOH solution (2×). Purification of the residue by FC (EtOAc/heptane 1:1→1:0) yielded the title compound (0.23 g, 71%). LC-MS: Rₜ=1.00 min, ES+=643.24.

### Biological Assay:

The following assay was carried out in order to determine the activity of the compounds of general formula (I) and their salts.

### In Vitro Assay:

### Inhibition of Human Recombinant Renin by the Compounds of the Invention:

The enzymatic in vitro assay was performed in 384-well polypropylene plates (Nunc). The assay buffer consisted of 10 mM PBS (Gibco BRL) including 1 mM EDTA and 0.1% BSA. The incubates were composed of 50 μL per well of an enzyme mix and 2.5 μL of renin inhibitors in DMSO. The enzyme mix was premixed at 4° C. and consists of the following components:
human recombinant renin (0.16 ng/mL)
synthetic human angiotensin (1-14) (0.5 μM)
hydroxyquinoline sulfate (1 mM)

The mixtures were then incubated at 37° C. for 3 h.

To determine the enzymatic activity and its inhibition, the accumulated Ang I was detected by an enzyme immunoassay (EIA) in 384-well plates (Nunc). 5 μL of the incubates or standards were transferred to immuno plates which were previously coated with a covalent complex of Ang I and bovine serum albumin (Ang I-BSA). 75 μL of Ang I-antibodies in assay buffer above including 0.01% Tween 20 were added and a primary incubation made at 4° C. overnight. The plates were washed 3 times with PBS including 0.01% Tween 20, and then incubated for 2 h at rt with an antirabbit-peroxidase coupled antibody (WA 934, Amersham). After washing the plates 3 times, the peroxidase substrate ABTS (2.2′-azino-di-(3-ethyl-benzthiazolinsulfonate), was added and the plates incubated for 60 min at rt. After stopping the reaction with 0.1 M citric acid pH 4.3 the plate was evaluated in a microplate reader at 405 nm. The percentage of inhibition was calculated of each concentration point and the concentration of renin inhibition was determined that inhibited the enzyme activity by 50% (IC₅₀).

### In Vivo Assay:

Compounds of the present invention may be tested according to the method described by Schnell et al. (Am. J. Physiol. 264 (Heart Circ. Physiol. 33), 1993, H1509-H1516).

1. Novel tetrahydropyridine derivatives according to formula (I) 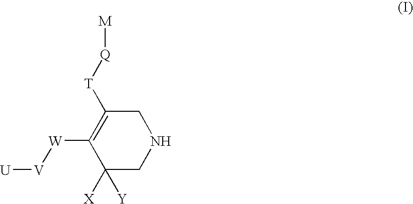
wherein
X and Y represent independently hydrogen, fluorine or a methyl group; X and Y do not represent both hydrogen at the same time or X and Y may together form a cyclopropyl ring;
W represents a phenyl or heteroaryl ring, the heteroaryl ring being a six-membered and non-fused ring, the phenyl ring and the heteroaryl ring are substituted with V in position 3 or 4;
V represents —(CH₂)ᵣ—; -A-(CH₂)ₛ—; —CH₂-A-(CH₂)ₜ—; —(CH₂)ₛ-A-; —(CH₂)₂-A-(CH₂)ᵤ—; -A-(CH₂)ᵥ—B—; —CH₂—CH₂—CH₂-A-CH₂—; -A-CH₂—CH₂—B—CH₂—; —CH₂-A-CH₂—CH₂—B—; —CH₂—CH₂—CH₂-A-CH₂—CH₂—; —CH₂—CH₂—CH₂—CH₂-A-CH₂—; -A-CH₂—CH₂—B—CH₂—CH₂—; —CH₂-A-CH₂—CH₂—B—CH₂—; —CH₂-A-CH₂—CH₂—CH₂—B—; —CH₂—CH₂-A-CH₂—CH₂—B—; —O—CH₂—CH(OCH₃)—CH₂—O; —O—CH₂—CH(CH₃)—CH₂—O—; —O—CH₂—CH(CF₃)—CH₂—O—; —O—CH₂—C(CH₃)₂—CH₂—O—; —O—CH₂—C(CH₃)₂—O—; —O—C(CH₃)₂—CH₂—O—; —O—CH₂—CH(CH₃)—O—; —O—CH(CH₃)—CH₂—O—; —O—CH₂—C(CH₂CH₂)—O— or —O—C(CH₂CH₂)—CH₂—O—;
A and B independently represent —O—; —S—; —SO— or —SO₂—;
U represents aryl or heteroaryl;
T represents —CONR¹—; —(CH₂)ₚOCO—; —(CH₂)ₚN(R¹)CO—; —(CH₂)ₚN(R¹)SO₂—; —COO—; —(CH₂)ₚOCONR¹— or —(CH₂)ₚN(R²)CONR¹—;
R¹ and R² independently represent hydrogen; lower alkyl; lower alkenyl; lower alkinyl; cycloalkyl; aryl-lower alkyl, heteroaryl-lower alkyl or cycloalkyl-lower alkyl;
Q represents lower alkylene or lower alkenylene;
M represents hydrogen; cycloalkyl; aryl; heterocyclyl or heteroaryl;
p is the integer 1, 2, 3 or 4;
r is the integer 3, 4, 5, or 6;
s is the integer 2, 3, 4 or 5;
t is the integer 1, 2, 3 or 4;
u is the integer 1, 2 or 3;
v is the integer 2, 3 or 4;
and in any form including optically pure enantiomers, mixtures of enantiomers such as racemates, diastereomers, mixtures of diastereomers, diastereomeric racemates, mixtures of diastereomeric racemates, and the meso-form; as well as in free acid or base form or pharmaceutically acceptable salts, solvent complexes and morphological forms.

2. Tetrahydropyridine derivatives according to claim 1 wherein X, Y, V, W and U are as defined in general formula (I); T represents —CONR¹—; Q represents lower alkylene and M represents hydrogen, aryl or heteroaryl.

3. Tetrahydropyridine derivatives according to claim 1 wherein X, Y, W, T, Q and M are as defined in general formula (I), V represents —CH₂CH₂O—; —CH₂CH₂CH₂O—; —OCH₂CH₂O— or —CH₂CH₂CH₂OCH₂O— and U is as defined in general formula (I).

4. Tetrahydropyridine derivatives according to claim 1 wherein X, Y, V, U, T, Q and M are as defined in general formula (I) and W represents a phenyl substituted in −4 position with V.

5. Tetrahydropyridine derivatives according to claim 1 wherein W, V, U, T, Q and M are as defined in general formula (I) and X and Y together may form a cyclopropyl group.

6. The compounds according to claim 1 selected from the group consisting of:
8-{4-[3-(2-Chloro-3,6-difluorophenoxy)propyl]phenyl}-5-aza-spiro[2.5]oct-7-ene-7-carboxylic acid cyclopropyl-(2,3-dichlorobenzyl)amide;
4-{4-[3-(2-Chloro-3,6-difluorophenoxy)propyl]phenyl}-5,5-dimethyl-1,2,5,6-tetrahydropyridine-3-carboxylic acid cyclopropyl-(2,3-dichlorobenzyl)amide;
4-{4-[3-(2-Chloro-3,6-difluorophenoxy)propyl]phenyl}-5,5-dimethyl-1,2,5,6-tetrahydro-pyridine-3-carboxylic acid cyclopropyl-(2-methoxy-3-methylpyridin-4-ylmethyl)amide;
8-{4-[3-(2-Chloro-3,6-difluorophenoxy)propyl]phenyl}-5-aza-spiro[2.5]oct-7-ene-7-carboxylic acid cyclopropyl-(2-methoxy-3-methylpyridin-4-yl-methyl)-amide;
8-{4-[3-(2-chloro-3,6-difluorophenoxy)propyl]phenyl}-5-azaspiro[2.5]oct-7-ene-7-carboxylic acid cyclopropyl-[2-(2-hydroxypropoxy)-3-methylpyridin-4-ylmethyl]amide;
4-{4-[3-(2-chloro-3,6-difluorophenoxy)propyl]phenyl}-5,5-dimethyl-1,2,5,6-tetrahydro-pyridine-3-carboxylic acid cyclopropyl-[2-(2-hydroxypropoxy)-3-methylpyridin-4-ylmethyl]amide;
4-{4-[3-(2-chloro-3,6-difluorophenoxy)propyl]phenyl}-5,5-difluoro-1,2,5,6-tetrahydropyridine-3-carboxylic acid cyclopropyl-(2,3-dichlorobenzyl)amide.

7. A pharmaceutical composition containing at least one compound according to claim 1 and pharmaceutically acceptable carrier material or adjuvants.

8. (canceled)

9. A method for the treatment or prophylaxis of diseases which are related to hypertension, congestive heart failure, pulmonary hypertension, renal insufficiency, renal ischemia, renal failure, renal fibrosis, cardiac insufficiency, cardiac hypertrophy, cardiac fibrosis, myocardial ischemia, cardiomyopathy, glomerulonephritis, renal colic, complications resulting from diabetes such as nephropathy, vasculopathy and neuropathy, glaucoma, elevated intra-ocular pressure, atherosclerosis, restenosis post angioplasty, complications following vascular or cardiac surgery, erectile dysfunction, hyperaldosteronism, lung fibrosis, scleroderma, anxiety, cognitive disorders, complications of treatments with immunosuppressive agents, and other diseases known to be related to the renin-angiotensin system, comprising the administration to a patient of a pharmaceutically active amount of a five-membered heteroaryl derivative according to claim 1.

10. (canceled)

11. The compound according to claim 1 which is 8-{4-[3-(2-Chloro-3,6-difluorophenoxy)propyl]phenyl}-5-aza-spiro[2.5]oct-7-ene-7-carboxylic acid cyclopropyl-(2,3-dichlorobenzyl)amide.

12. The compound according to claim 1 which is 4-{4-[3-(2-Chloro-3,6-difluorophenoxy)propyl]phenyl}-5,5-dimethyl-1,2,5,6-tetrahydropyridine-3-carboxylic acid cyclopropyl-(2,3-dichlorobenzyl)amide.

13. The compound according to claim 1 which is 4-{4-[3-(2-Chloro-3,6-difluorophenoxy)propyl]phenyl}-5,5-dimethyl-1,2,5,6-tetrahydro-pyridine-3-carboxylic acid cyclopropyl-(2-methoxy-3-methylpyridin-4-ylmethyl)amide.

14. The compound according to claim 1 which is 8-{4-[3-(2-Chloro-3,6-difluorophenoxy)propyl]phenyl}-5-aza-spiro[2.5]oct-7-ene-7-carboxylic acid cyclopropyl-(2-methoxy-3-methylpyridin-4-yl-methyl)-amide.

15. The compound according to claim 1 which is 8-{4-[3-(2-chloro-3,6-difluorophenoxy)propyl]phenyl}-5-azaspiro[2.5]oct-7-ene-7-carboxylic acid cyclopropyl-[2-(2-hydroxypropoxy)-3-methylpyridin-4-ylmethyl]amide.

16. The compound according to claim 1 which is 4-{4-[3-(2-chloro-3,6-difluorophenoxy)propyl]phenyl}-5,5-dimethyl-1,2,5,6-tetrahydro-pyridine-3-carboxylic acid cyclopropyl-[2-(2-hydroxypropoxy)-3-methylpyridin-4-ylmethyl]amide.

17. The compound according to claim 1 which is 4-{4-[3-(2-chloro-3,6-difluorophenoxy)propyl]phenyl}-5,5-difluoro-1,2,5,6-tetrahydropyridine-3-carboxylic acid cyclopropyl-(2,3-dichlorobenzyl)amide.
